(19) United States
(12) Patent Application Publication
(10) Pub. No.: US 2002/0155069 A1
(43) Pub. Date: Oct. 24, 2002

(54) Cosmetic treatment and device

(76) Inventors: Francis Pruche, Senlis (FR); Serge Forestier, Claye-Souilly (FR); Michel Kermici, Paris (FR)

Correspondence Address: Nixon & Vanderhye, 8th Floor, 1100 North Glebe Road, Arlington, VA, 22201-4714, US

(21) Appl. No.: 10/069,460

(22) PCT Filed: Jun. 22, 2001
(86) PCT No.: PCT/FR01/01983

(30) Foreign Application Priority Data
Jun. 26, 2000 (FR) 00/08176

(51) Int. Cl. A61K 7/00

(52) U.S. Cl. 424/47

(57) ABSTRACT

Process and device for treating, especially for making up, for coloring or for the cosmetic care of some or all of the human or animal body The process for treating, especially for caring for, making up or coloring, at least a part of the human or animal body, is carried out by applying at least two treatment products simultaneously to a localized area of said part of the body as a function of characteristics of the localized area and/or of a predetermined design, in order to obtain a mixture of said treatment products directly on the localized area, at least one treatment product comprising at least one solvent in a proportion of at least 10% by weight.

The present invention relates to the treatment, especially the making up, coloring or cosmetic care of some or all of the human or animal body.

The technique of tattooing has been known since ancient times and consists in indelibly inserting coloring substances under the epidermis.

Tattooing makes it possible to obtain a color design of very high quality, but requires a delicate intervention, in particular in terms of the health risk on account of the incision of the skin. As a result, many people abandon the idea of being tattooed on account of the indelibility and the surgical aspect of the operation.

Make-up products which offer temporarily a certain covering effect and which can substantially modify the appearance of the part of the body they cover are also known.

Make-up products are easy to apply, but they do not stay on for more than a few hours, especially in hot and/or humid weather.

Moreover, make-up products seek to imitate the appearance of normal skin and do not offer any freedom of design.

Small stamping blocks for applying a design onto the skin by transfer printing have recently appeared. The durability of the design on the skin is of the order of a few days. However, no freedom of design is offered. The user merely reproduces the design present on the stamping block.

Document U.S. Pat. No. 5,268,166 also discloses an application system for applying a mixture of colored cosmetic materials directly onto the skin by electrostatic spraying. However, the cosmetic materials must be suitable for electrostatic spraying, that is to say that they must be able to be electrically charged and designs cannot be created on account of the excessively coarse definition due to the large drop sizes required for an acceptable speed of treatment.

The present invention proposes to overcome the drawbacks mentioned above.

The present invention proposes a process for treating, especially for caring for, making up or temporarily coloring, without incision of the skin, offering total freedom of design and freedom in the choice of products.

The treatment, especially the care, make-up or coloring process according to one aspect of the invention is intended for at least a part of the human body. The process involves the application of at least two treatment products simultaneously or sequentially onto a localized area of said part of the human body as a function of characteristics of the localized area and/or of a predetermined design, in order to obtain a mixture of said treatment products directly on the localized area, preferably at least one treatment product comprising at least one solvent in a proportion of at least 10% by weight.

The expression “part of the animal or human body” means herein the skin, for example of the face, of a limb, etc., the scalp, mucous membranes, semi-mucous membranes, keratin fibers, for example the eyelashes, the eyebrows, head hair and other hairs, and also the nails.

The expression “characteristics of the localized area” means herein:

for the skin, the color and the dry, greasy or combination nature;

for the hair, the color, the thickness, the diameter and the damaged, cracked or split state;

for the nails, the color and the hard, soft, crazed or brittle state.

Advantageously, the application of several treatment products is performed by jet.

In one embodiment of the invention, at least one jet is produced by a thermal means capable of forming bubbles of treatment product.

In another embodiment of the invention, at least one jet is produced by a piezoelectric means.

In one embodiment of the invention, at least one jet is diverted.

The application precision of the process allows a treatment of a localized area of small size, for example around a hair in order to achieve an injection into the root of the hair of droplets of care product, of an anti-greasy type or of a type promoting hair growth, such as Minoxidil®. A precise treatment of a comedone may also be achieved by means of a bactericidal product. The application of active product to sites at which their action is inoperative or needless is thus avoided, which is economical and more pleasant for the user.

In one embodiment of the invention, a step of viewing a chosen coloration or make-up design takes places before the treatment products are applied.

The invention also proposes a device for carrying out the above process. The device comprises a means for positioning said part of the human body, and a means for applying treatment products to said part as a function of a predetermined design.

Advantageously, the application means comprises a plurality of treatment product reservoirs and a plurality of spraying nozzles, each nozzle being fed by a reservoir.

Advantageously, the application means comprises at least two, in particular 3 or 4, or even five reservoirs and/or at least two, in particular 3 or 4, or even five nozzles.

In one embodiment of the invention, the application of treatment products is carried out by a moving head which follows the relief of the part while remaining a short distance away or being in contact. The moving application head may comprise a powder puff, a roll or a coarse or fine brush, usually used in cosmetics.

The distance between the moving application head and said part may be monitored in real time. Monitoring of the local characteristics obtained immediately after the application of the products may be carried out by means of the mobile application head. The monitoring may be carried out by feedback or automatic control, with real-time correction of the make-up operation or of the treatment, if necessary.

The flow rate of each product may be variable as a function of the coordinates of the site onto which said product is being applied. A step of recognition of said part may be carried out in order to determine what organ said part belongs to, for example the arm, the hand, the scalp, the face and its parts such as the eye, the eyelid, the cheek, the eyelash, the eyebrow, the lip, the forehead or the nose.

A three-dimensional reconstruction of said part may be envisaged in order to obtain a two- or three-dimensional and preferably a three-dimensional representation. The three-dimensional representation is produced in particular from at least two pictures.

The device may comprise a means for positioning said part of the human body, for example in the form of a cast or a chinstrap intended to immobilize the part to be treated (arm, head, etc.).

The image analysis means may be software which knows, in principle, the position or shape of the part to be treated (for example the lips) , in particular which processes the image by regional growth.

In one embodiment of the invention, the moving application head is supported by an articulated arm in order to be able to follow the relief of said part.

A light source may be combined with the image-taking means.

The application means may be supported by an articulated arm and may comprise a nozzle, for example of piezoelectric type.

The device may comprise means for controlling the position of the application head relative to the part of the area to be treated.

The device may comprise a means for automatic control of the total amount and of the respective amounts of each composition and of each ingredient as a function of the desired characteristics, in particular with feedback.

An efficient application machine which allows a non-invasive tattoo to be produced easily is thus provided.

Thus, the invention offers the advantages of freedom of design of the traditional tattoo, the ease of use and health safety of the make-up operation and an average durability which may be readily controlled according to the wishes of the user, from a few hours to about twenty days. The fact that the local characteristics of said part are taken into account further increases the visual quality of the design obtained, in particular to play on optical or perspective or three-dimensional effects, enabling certain skin imperfections to be made invisible.

The term “image” means herein a two-dimensional representation of the appearance of an object composed of unit elements to be analyzed; the analysis area may be limited to a single element (for example a pixel).

The present invention will be understood more clearly by studying the detailed description of a number of embodiments taken as examples that are in no way limiting and illustrated by the attached drawings, in which:

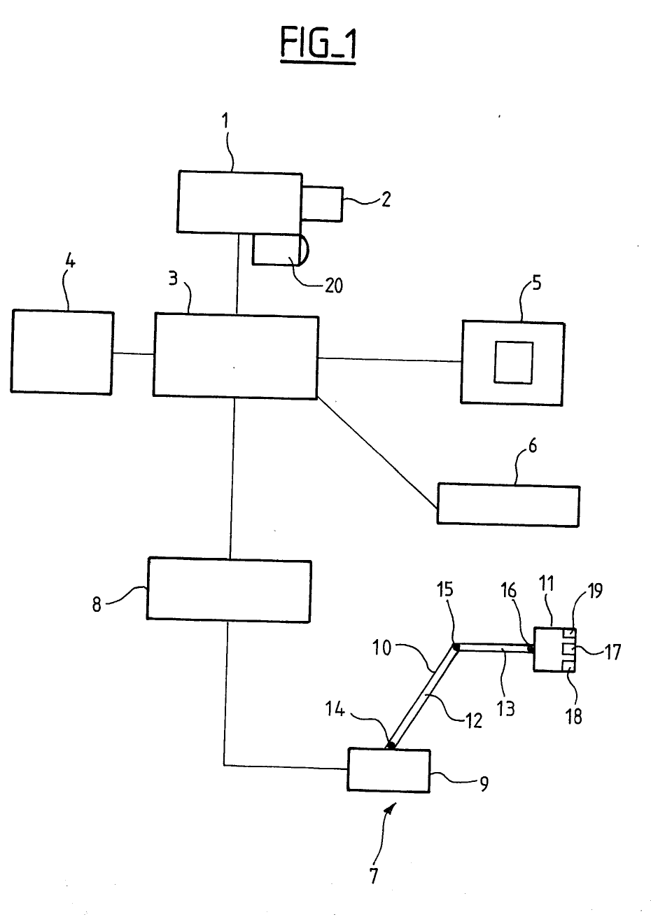

FIG. 1 is a diagrammatic view of a device according to one embodiment of the invention;

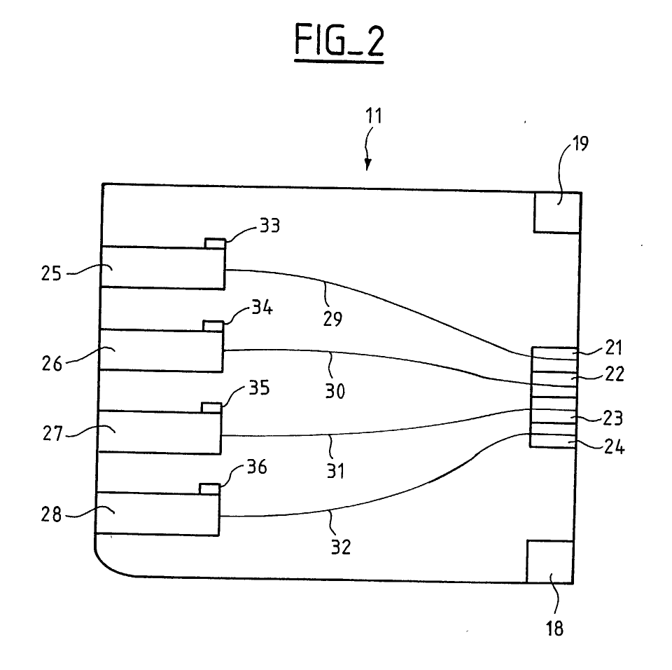

FIG. 2 is a detailed view of FIG. 1;

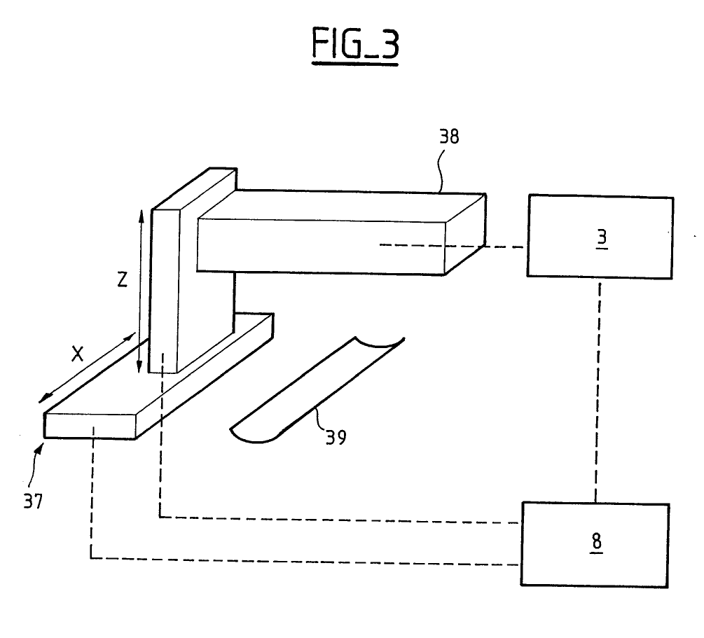

FIG. 3 is a diagrammatic view according to another embodiment of the invention; and

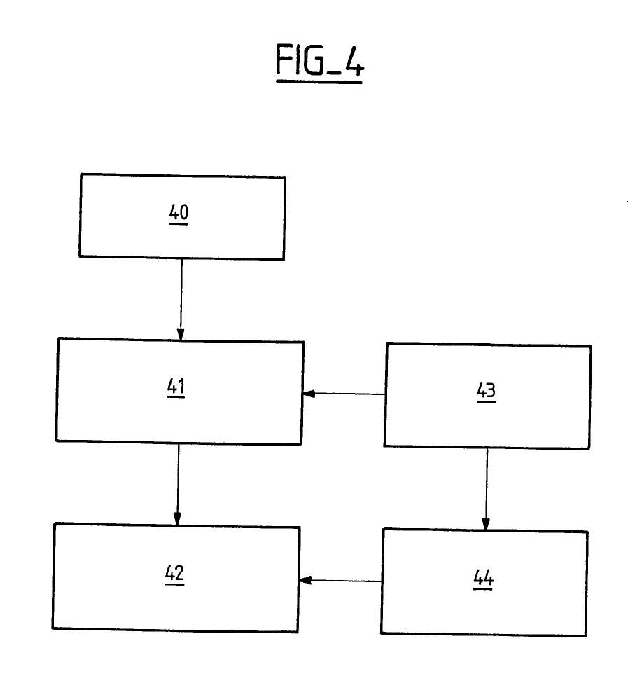

FIG. 4 is a diagram showing the image acquisition steps in a device in accordance with the invention.

As may be seen in FIG. 1, the coloring or make-up system comprises at least one camera 1 equipped with an objective 2, a central processing unit 3 equipped with a memory 4, a screen 5 and a keyboard 6, and an applicator 7 provided with control means 8. The video camera 1 may be of CCD type. The communication between these various elements may be provided by a connection of RS 232 type. The memory 4 and the screen 5 may be integral to the central processing unit or arranged in separate cases. The presence of the keyboard 6 is optional and may be replaced with a screen 5 of touch type allowing commands to be made. A mouse or a device of the same type may also be provided.

The applicator 7 comprises a case 9 which may be fixed to the floor or to any suitable support, an articulated assembly 10 fixed at one end to the case 9 and supporting at the opposite end a product application head 11.

The articulated assembly 10 comprises two arms 12 and 13. The arm 12 is pivotably mounted on the case 9 by means of an articulation 14. The arm 13 is pivotably mounted on the arm 12 by means of an articulation 15 and the head 11 is pivotably mounted on the arm 13 by means of an articulation 16. The articulations 14, 15 and 16 are motorized or provided with actuators for moving the head 11 relative to the case 9 along several axes, preferably perpendicular to each other. Optionally, the arms 12 and 13 may be telescopic, for example by means of an electric jack. If it is desired for the head 11 to make more complex movements, more than three articulations may be provided so that they give said head 11 a larger number of degrees of freedom.

A light source 20 may be attached to the camera 1 to improve the lighting and thus the quality of the images obtained. The light source 20 will be active at least in visible light and may be of electroluminescent diode, xenon arc, halogen, etc., type.

The application head 11 comprises a row of spraying nozzles 17 fed with treatment products from one or more reservoirs, not shown, for example arranged in the case 9, and two distance sensors 18 and 19 capable of measuring the distance between the application head 11 and the surface onto which the treatment product is to be applied. The detectors 18 and 19 may each include a laser diode emitting a laser beam adjusted to cross the other laser beam emitted by the other diode at the desired distance between the head 11 and the surface which is to receive the treatment product, such that a difference relative to this desired distance can be readily detected. The nozzles 17 may be of the inkjet type with a piezoelectric crystal.

Inkjet printing is a contactless method. The ink is emitted from nozzles. Liquid inks of different colors spurt onto the surface to be treated to form an image. The application head 11 sweeps over said surface in parallel strips. To increase the printing speed, the application head 11 prints in one pass a row of pixels simultaneously by means of the row of nozzles 17. The inkjet technique is generally either thermal or electrostatic, or even piezoelectric.

In the present application, the piezoelectric technique in which a piezoelectric crystal is placed in the bottom of a product reservoir close to a nozzle is preferably used. When a current is applied to the piezoelectric crystal, it becomes deformed, which creates a force sufficient to eject a droplet of product. The product does not need to be heated and the droplets may be of very small size. In order to obtain color designs, cyan, magenta and yellow color product cartridges will be provided. Preferably, a black cartridge will also be provided in order to obtain a good-quality black. One to four additional cartridges, or even more, may also be provided, for example for white, light cyan, light magenta, and metalized colors (gold or silver) for finer designs.

Needless to say, the characteristics of the product besides the color characteristics, will be adapted to the part of the human body intended to receive them: skin, nails, hair, etc. Moreover, cartridges of care product and/or of make-up product will also be provided.

The system functions as follows. A person wishing to treat a part of his body, for example the face, the hand, the hair, etc., is installed in the field of vision of the camera 1 for one or more views to be taken. Specifically, the production of a three-dimensional image of the part of the human body, which is a preferred variant, requires at least two views to be taken at different angles by means of at least two fixed cameras or by means of a moving camera. The image files obtained on taking these views are transferred from the camera 1 to the central processing unit 3 which performs processing generally known as reconstruction, allowing a three-dimensional image to be obtained which is then transferred to the memory 4. To improve the quality of the three-dimensional image, optional processing may be performed by the central processing unit, for example to correct geometrical defects associated with the perception of the relief. The three-dimensional image obtained and stored in the memory 4 may be displayed on the screen 5. In the case of a two-dimensional image, it is not essential for the central processing unit 3 to perform such processing.

Design software stored in the memory 4 allows the user or an operator who may assist him to simulate on the screen 5 various possible make-ups or colorations as a function of preexisting designs stored in the memory 4, or of designs provided by the user on a digital medium such as a floppy disk or a CD ROM capable of being read by a suitable reader, not shown, connected to the central processing unit 3. Retouching may be performed by means of the keyboard 6 or the screen 5 if the latter is a touch screen, until the effect desired by the user is obtained on the screen 5. The design may also be entirely composed by the user or by the operator. Design software such as PHOTOSHOP® from the company Adobe or PAINTBRUSH® from the company Microsoft may be used.

At the same time that this choice is made by the user, the central processing unit performs an analysis of the characteristics of the surface to be made up or colored from the two- or three-dimensional image, so as to determine the characteristics, especially the topological characteristics, of said surface, in order to determine whether an application of a treatment product is necessary prior to the application of a coloring or make-up product, for example in the case of a dry skin or in the case of a wrinkled skin. The two-dimensional image is suitable for dry skin or nonuniform colors (pigmentation mark or scar) . For wrinkled skin, the three-dimensional image is preferred.

The central processing unit 3 also performs processing to allow different parts of the human body to be recognized, in particular to differentiate hair from skin, to recognize the fingers and the nails of a hand and to recognize the various parts of the face, in particular the lips, the eyebrows, the cheeks and more generally any part requiring the application of a specific treatment, make-up or coloring product, for example by means of segmentation-recognition software.

At the end of these steps, the application head 11 is brought into the field of vision of the camera 1 and close to the surface which is to be made up or colored, such that the row of nozzles 17 is at the desired spraying distance, which is checked by means of the sensors 18 and 19. Needless to say, the part of the user's body which is to be treated may, preferably, be immobilized throughout the treatment; however, it is possible to add a means allowing said part of the human body to be followed, if it moves. The dynamic control of the head-surface distance is performed in real time by the control means 8.

Two or more products are simultaneously deposited in order to accelerate the process. To this end, several rows of nozzles 17 arranged in a matrix are provided, allowing different products to be applied simultaneously to the same place (localized area). In the event of scars being present, they may be camouflaged by applying colorant with optical illusion patterns, making it possible to give an illusion of relief. The central processing unit 3 will perform a colorimetric analysis of the image perceived by the camera 1 so as to determine the local shade of the part which is to be treated.

Several products are applied simultaneously to form a mixture in situ. This thereby avoids the use of an excessive amount of products whose mixture is specific to a person or to a localized area of a person and cannot be used elsewhere. The mixture is produced specifically in real time and on the site at which it is to be applied.

A coloring product, for example an ink, may be applied in order to obtain the image selected by the user on the screen 5. A step of applying a varnish and/or a product intended to regulate the desquamation and enabling the staying power of the coloration to be prolonged may then be included. Needless to say, an ink and optionally a varnish which can be removed without damaging the skin, for example by means of an adequate make-up remover, such as an organic solvent, water and/or a surfactant product, will be selected.

In the case of coloring or making up a bald head or a head on which the hair may be cut extremely short, products intended for the skin to produce the pattern desired by the user, such as streaks, gradations, stripes, etc. will be used.

The spraying nozzles 17 may be of the piezoelectric spraying type, allowing a wide variety of treatment products to be used. The product is forced through the nozzle which is of small diameter and made to vibrate at high frequency by a piezoelectric crystal placed in the head 11. The product in liquid form then splits into fine droplets which are expelled by the nozzle. At the outlet, the droplets may be diverted by any known means such as by means of deflection electrodes, allowing a multideflected continuous jet printing.

The entire surface to be made up or colored is crossed by the head 11 with real-time measurement of the distance by means of the sensors 18 and 19 for maintaining the distance, which are required by the type of nozzle used, thus allowing a precise monitoring of the relief and a high-quality application.

In one embodiment of the invention, the camera 1, the applicator 7, and a screen are arranged in a first location, and a data processing unit equipped with its peripherals is arranged in a second location and is connected to the camera 1 and the applicator 7 by means of a communication network, for example of Internet type. Interfaces such as modems will be connected to the camera 1 and the applicator 7 on the one hand, and to the data processing unit on the other hand. The data processing unit may be combined with a database which allows a larger choice of designs. Moreover, the data processing unit may be of high calculating power. The data processing unit sends one or more images to the first location where the user selects an image and indicates his approval. The production of the control signal may then be carried out at the first location by a local data processing means, or at the second location by said data processing unit on receiving a signal representative of the user's choice.

As a variant, the user can select his image at a third location, for example at home, on a personal computer connected to a communication network, indicate his approval on an image, this approval then being transmitted to said data processing unit which then produces a signal intended to control the applicator, and sends it. The applicator, on receiving said signal, is ready to produce the image. The user then goes from the third location to the second location, for example a beauty salon, a manicure salon, a pharmacy, etc.

Alternatively, the user has the applicator at home. He can also have the camera at home. In this case, the image taken by the camera is sent to a remote data processing unit which performs the various operations required, proposes at least one image, and produces and sends a signal intended to control the applicator on receiving the user's approval.

FIG. 2 illustrates the moving head 11 in greater detail. The set of nozzles 17 comprises four nozzles 21, 22, 23 and 24, four removable cartridges 25, 26, 27 and 28, each containing a product that it is desired to apply and being connected via a tube 29, 30, 31, 32 to the corresponding nozzle 21, 22, 23, 24. For example, the cartridges 25 to 28 can contain:

each a base coloring composition

each a coloring composition derived from a mixture;

one a covering product of the foundation type, and the others different coloring compositions, etc. A number of cartridges greater than four may be provided for a wider choice of compositions.

The moving head 11 comprises a means for identifying the cartridges 25 to 28, for example in the form of four sensors 33 to 36, each dedicated to a cartridge 25 to 28 and being capable of recognizing the content of the cartridge, in particular by reading a mechanical, optical, magnetic, etc. code.

Product pumping means are also provided to transfer a product from a cartridge 25, 26, 27, 28 to the corresponding nozzle 21, 22, 23, 24. The moving head 11 may comprise a means for monitoring the amount of product present in each cartridge 25, 26, 27, 28, for example in the form of a sensor dedicated to measuring the mass of a cartridge 25, 26, 27, 28 and allowing an estimation of the level of product, or a sensor dedicated to measuring the flow rate of a pumping means, or alternatively a sensor dedicated to measuring the electrical current consumed by a pumping means, the current decreasing when a cartridge is empty and when the pumping means no longer delivers product. An empty cartridge may be indicated by displaying a “cartridge empty” message on the screen 5 and/or by emitting a sound signal. Alternatively, the screen 5 may comprise a zone dedicated to displaying the level of product in each cartridge.

The moving head 11 may comprise a temperature maintenance means if the nature of the products, in particular their viscosity, requires it, for example between 20 and 27° C. and better still between 22 and 24° C. Maintenance at a relatively constant temperature avoids a dispersion of flow from the nozzles due to a variation in viscosity.

Thus, after selecting the desired visual characteristics, the software stored in the memory 4 and used by the central processing unit 3 checks that the cartridges present in the head 11 are suitable for the desired visual characteristics. If such is not the case, a warning message is displayed on the screen 5.

The check is made from the signals emitted by the sensors 33 to 35 and received by the central processing unit 3, for example by a wireline connection passing via the case 9.

After the operator has made available, if necessary, suitable cartridges in the head 11, a corresponding message may be displayed on the screen 5.

The software calculates the instantaneous amounts of each product for each elementary area of the part to be treated as a function of the characteristics of said elementary area. In other words, for an elementary area of coordinates (x, y) or (x, y, z), the software calculates the partial elementary amounts Q₂₅, Q₂₆, Q₂₇ and Q₂₈ of products derived from cartridges 25, 26, 27 and 28 as a function of the type of each product, the characteristics of the elementary area and the result to be obtained which may be defined by color and brightness variables.

The software also determines the order of application of the products, which may be successive on the same area or juxtaposed on neighboring areas. In certain cases, only one product will be applied and only one cartridge will be used, even if others remain in place on the head 11.

In order to have a direct correlation between the visible characteristics of the skin and the two- or three-dimensional mathematical information, the software may use, for example, an optical measurement process which uses a combination of Gray code and phase shifting techniques. It is possible with this method to determine with great precision the absolute spatial coordinates of all the object points in the field covered by the image.

In the Gray code method, the fringes are projected successively with a rectangular luminosity modulation and a different number of lines. The number of lines is doubled at each projection process, thus unambiguously defining the order of the lines for each image point. In the phase shifting method, only one fringe is projected several times with a sinusoidal luminosity modulation and a different phase relationship. This also allows an exact three-dimensional reconstruction of the surface for which each image point is defined independently of its neighbors, and automatic control of the measuring quality.

The resolution in the vertical Z direction typically with 0.2% of the measuring field leads to an effective resolution of 4 μm in Z. Depending on the type of CCD camera used, a resolution of 45 μm will be achieved in the horizontal X and Y direction. The image analysis sequence with analysis of the corresponding coordinates may be performed in less than one second (typically 500-800 ms).

The three-dimensional area coordinates observed by the camera 1 serve to position the head 11 at a suitable distance (typically 1 cm) from the skin. This is performed by controlling a Z-translational displacement table by means of the central processing unit 3.

The image acquired by the camera 1 for calculating the three-dimensional coordinates of the area observed also makes it possible to measure the color of the skin. To do this, the camera 1 is calorimetrically calibrated as is done for a scanner using an image of a calibration test card and calibration software, for example Profile Maker from the company LOGO. To overcome skin brightness phenomena, crossed-positioned polarizers are used, which are placed on the projector 20 and in front of the camera objective. This calibration procedure makes it possible to obtain the correspondence of the image acquired with the colorimetric system and independent of the camera. The image makes it possible to have the color on each pixel and thus at each point of the area observed.

The positioning system 37, illustrated in FIG. 3, is composed of two translational displacement tables with stepping motors integrated together, driven via a control unit. These tables allow the displacement of the distribution head 38 in Z (distance to the application area) and in X (translational displacement along the application area).

The distance from the distribution head 38 to the area to be treated may range between 20 μm and 10 cm, preferably between 100 μm and 5 cm and preferably between 250 μm and 1 cm.

The area of the body to be treated is attached by means of a suitable device. Mention is made, for example, of a cast 39 for the arm and of a chinstrap, not shown, for the head.

Design software supporting the screen and printing colorimetric calibrations (of the type Photoshop V5.02 from the company ADOBE) makes it possible to select from an image database the type of make-up to be applied to the area and to visualize the result after application. The image acquired by the calibrated camera allows the exact restitution of the colors of the area.

By means of the image modification software, it is possible to selectively correct a defect in the area to be treated (example: depigmented area). This area is encircled using the mouse and can be corrected using the same color as the surrounding skin.

The colors are obtained by mixing together the four colors: cyan, magenta, yellow, black. The head is colorimetrically calibrated (ICC profile) so as to fully restore the colors of the make-up selected and viewed on the screen.

In one preferred mode of the invention, a dynamic control of the position of the distribution head 38 relative to the skin may be made by means of a telemetry system. For example, using a contactless distance sensor operating on the principle of triangulation. The sensor has a measuring dynamic of plus or minus 1 cm with a precision of less than 10 μm and a working distance of 6.5 cm, for example of the BULLIER INTERNATIONAL type, Reference M5L/20. The signal from the sensor is digital and is connected to the central processing unit. The automatic control of the position of the distribution head makes it possible to compensate for the small movements in the area to be treated. The automatic control is performed by the central processing unit during the printing of the selected design.

By means of the invention, a make-up operation, a coloration or a care treatment adapted to the user's wishes may be achieved. The expression “make-up operation” means herein the application of product taking into account the colorimetric and topological characteristics of the skin, and the term “coloration” means the application of covering product totally camouflaging the original shade of the skin. The system also applies to hair dyeing. Patterns in different colors and of different shapes may be produced thereon by means of this automatic treatment machine.

FIG. 4 illustrates the various steps of the image acquisition process, in particular in the case of two identical cameras looking at an object from two different optical views. The two images, called the left image and the right image, are acquired at the same time, since the two cameras are synchronized. The view-taking is immediate, which eliminates any problems of movement of the user. The working volume is limited by the size of the vertical and horizontal fields of the cameras and also by the depth of focus of the objectives. The two views show disparities which may be quantified, and which make it possible to return to the topography of the observed surface. The topography of the observed object is calculated in step 40 by acquisition of the left and right images, in step 41 by calculating the optical geometry with interpolation and erecting of the images performed by the central processing unit 3 of FIG. 1, and in step 42 of calculating the disparities between the left and right images and by calculating the topography of the surface.

By way of example, in step 43, the calibration data are obtained using a test card which is moved in the working volume of the two cameras, the calibration being performed on a hundred points per plane on several planes separated by a step, for example of a few millimeters depending on the object to be measured. Parameters extrinsic to the cameras relating to the positions and orientations relative to the calibration reference point, and parameters intrinsic to the cameras (optical characteristics) linked to the associated distortion and pin diaphragm model, are extracted therefrom. This calibration is performed once only and defines the geometry of the two cameras. The calculation of the disparities between the cameras is performed at two different scales. Next, the correlation minimum between the two views is searched for at the two scales described. The position of this minimum is interpolated parabolically, which gives a sufficient precision whose error is less than one pixel. The height of the point selected is inversely proportional to the calculated position of the minimum. The calibration data calculated in step 43 are supplied during step 41 of calculating the optical geometry and during a step 44 of calculating new parameters of the cameras, which are supplied to the central processing unit during step 42 of calculating the topography of the surface.

The treatment product which may be applied in the context of the present invention may be of any cosmetically acceptable nature.

It may be a care, make-up or coloring product, which may be applied to the skin of the body and/or the face, including the scalp, to head hair, the eyelashes, the eyebrows, other hairs and/or to the nails.

The treatment products according to the invention may especially comprise, in preferred embodiments, active agents, especially cosmetic or even therapeutic active agents, preferably chosen from:

hair dyeing agents such as natural or synthetic dyes, for direct dyeing, oxidation dyeing, reduction dyeing or enzymatic dyeing; mention may be made in particular of henna and its derivatives; hair bleaching agents, for example oxidation bleaching agents; mention may be made in particular of hydrogen peroxide and persalts;

temporary skin coloring agents; mention may be made in particular of self-tanning agents such as DHA (dihydroxyacetone);

desquamating agents;

skin depigmenting or bleaching agents; mention may be made in particular of hydroquinone derivatives such as hydroquinone, arbutin or kojic acid;

skin tensioning agents, in particular tensioning polymers;

film-forming or non-film-forming, synthetic or natural, hydrophilic, lipophilic or amphiphilic, ionic or nonionic polymers;

antimicrobial agents, in particular antibacterial or antifungal agents, such as Octopirox® or Irgasan®;

haircare active agents, in particular anti-greasy active agents; antidandruff active agents;

agents for regulating hair growth or hair loss, in particular Minoxidil;

moisturizers such as polyols, especially glycols, in particular glycerol;

skincare agents, such as antiacne agents, especially benzoyl peroxide; anti-greasy agents or matt-effect agents; retinoids; antiwrinkle agents; care products for pigmentation marks or depigmented areas;

agents for nourishing the skin, the hair or the nails, such as vitamins, sugars, amino acids, peptides, proteins and plant extracts, of terrestrial or animal origin;

vitamins, trace elements;

sugars;

amino acids, peptides, proteins;

plant extracts of terrestrial or marine origin;

dyestuffs such as water-soluble or liposoluble dyes and/or pigments.

The pigments may be white or colored, mineral and/or organic, and micrometer-sized or nanometer-sized.

Among the mineral pigments and nanopigments which may be mentioned are titanium oxide, zirconium oxide or cerium oxide, and also zinc oxide, iron oxide or chromium oxide, ferric blue, chromium hydrate and ultramarines (aluminosilicate polysulfides).

Mention may also be made of nacreous pigments and interference pigments.

Among the organic pigments which may be mentioned are carbon black and lakes such as calcium, barium, aluminum, zirconium or strontium salts.

Among the dyes which may be mentioned are dyes that are common in the field under consideration, such as azo dyes, anthraquinone dyes or heterocyclic dyes; mention may be made of the disodium salt of ponceau, the disodium salt of alizarin green, quinoline yellow, the trisodium salt of amaranth, the disodium salt of tartrazine, the monosodium salt of rhodamine, the disodium salt of fuchsin, and xanthophyll, and mixtures thereof.

The following may also be provided:

antioxidants such as vitamin E, vitamin C, glutathione or glutathione esters;

permanent-waving agents for the hair, such as thioglycolic acid and its salts, in particular glyceryl thioglycolate;

slimming agents;

antioxidants or free-radical scavengers, such as [lacuna]

mineral or organic, lipophilic or hydrophilic sunscreens;

ceramides;

metal-complexing agents such as ETDA or its salts.

The treatment products may also comprise at least one solvent capable of dissolving or dispersing said active agents. This solvent may be polar or apolar, lipophilic or hydrophilic, and miscible or immiscible with water. It is preferably volatile enough to allow good adhesion of the active agent to the stratum corneum, or good penetration into keratin fibers. It is preferably in the form of a medium that is liquid at 15-35° C., and allows the solubilization or dispersion of the active agents. It may have a vapor pressure of from 40 to 45 mbar measured at 20° C. and/or a boiling point of between 30° C. and 120° C.

Solvents which may be used in particular, alone or as a mixture, include water; alcohols, in particular C₁-C₆ alcohols such as ethanol or isopropanol; glycols such as ethylene glycol or propylene glycol; propylene glycol ethers; ketones; esters; ethers; hydrocarbons and in particular isoparaffins; cyclic aromatic compounds (toluene, benzene and xylene); aldehydes; halogenated and in particular fluorinated hydrocarbon-based compounds; silicone compounds; physiologically acceptable oils, in particular volatile oils.

Among the volatile or nonvolatile oils which may be mentioned are:

hydrocarbon-based oils of animal origin such as perhydrosqualene;

hydrocarbon-based plant oils such as liquid triglycerides of fatty acids of from 4 to 10 carbon atoms, for instance heptanoic or octanoic acid triglycerides; sunflower oil, corn oil, soybean oil, marrow oil, grapeseed oil, groundnut oil, sweet almond oil, beauty-leaf oil, palm oil, sesame oil, hazelnut oil, apricot oil, macadamia oil, castor oil, avocado oil; caprylic/capric acid triglycerides; jojoba oil, karite butter;

liquid paraffins and derivatives thereof, petroleum jelly, polydecenes and hydrogenated polyiosbutene (parleam);

synthetic esters and ethers, in particular of fatty acids, for instance the oils of formula R₃COOR₄ in which R₃ represents a higher fatty acid residue containing from 7 to 29 carbon atoms and R₄ represents a hydrocarbon-based chain containing from 3 to 30 carbon atoms, such as, for example, purcellin oil, isopropyl myristate, 2-ethylhexyl palmitate, 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate, triisocetyl citrate and fatty alkyl heptanoates, octanoates or decanoates; polyol esters, for instance propylene glycol dioctanoate, neopentyl glycol diheptanoate or diethylene glycol diisononanoate; and pentaerythritol esters;

the fatty alcohols containing from 12 to 26 carbon atoms, for instance octyldodecanol, 2-butyloctanol, 2-hexyldecanol, 2-undecylpentadecanol and oleyl alcohol;

partially hydrocarbon-based and/or silicone-based fluoro oils;

silicone oils, for instance volatile or nonvolatile, linear or cyclic polydimethylsiloxanes (PDMS); alkyldinethicones; silicones modified with aliphatic and/or aromatic groups, that are optionally fluorinated, or with functional groups such as hydroxyl, thiol and/or amine groups; phenylsilicone oils such as polyphenylmethylsiloxanes or phenyltrimethicones;

volatile oils (oil having a measurable vapor pressure at 25° C. and 1 atmosphere, for example greater than 0 Pa, in particular ranging from 10⁻³ to 300 mmHg (0.13 Pa to 40,000 Pa)); mention may be made of volatile silicone oils, such as cyclic or linear volatile silicones, and cyclocopolymers. Mention may also be made of volatile hydrocarbon-based oils such as isoparaffins, and volatile fluoro oils.

Preferably, the treatment product comprises from 0.001% to 25% by weight of active agent, in particular from 0.01% to 15% and better still from 1% to 10%.

Moreover, preferably, at least one of the products, and better still all the products, comprises a solvent in a proportion of at least 10% by weight, in particular at least 15-80% by weight and preferably 20-60% by weight.

Thus, in one preferred embodiment, at least two treatment products may be applied to the skin, one of which comprises α-hydroxy acids and the other of which comprises kojic acid; a skin depigmenting product is thus obtained.

In the case of a pigmentation mark or a depigmented area (for example of vitiligo type) detected by the camera 1 and located by the central processing unit 3, the treatment product may be or may comprise a covering product to give the pigmentation mark the same appearance as the rest of the skin, for example a product comprising glycerol and a pigment, in particular an iron oxide.

The care products used prior to the make-up or coloring product may also be used by mixing in situ a dye and a care product at low dose, such as hydroquinone, kojic acid or arbutin. It is also possible to add in situ, independently of or simultaneously with the coloring or make-up product, a self-tanning agent, for example dihydroxyacetone, or alternatively a combination of substrate and enzyme reacting in situ on the skin, the hair or the nails during the application, the substrate possibly being of the polyphenol family, in particular a flavonoid or a hydroxystilbene.

In another preferred embodiment, at least two treatment products may be applied to the hair, one of which comprises an antifungal agent (such as Octopirox), and the other of which comprises an agent for preventing hair loss (such as Aminexyl); a haircare product is thus obtained.

A combination of a base, a coupler and an oxidizing agent, usually used in the field of hair dyeing, may also be applied. Moreover, the oxidizing agent may be replaced with an enzyme and a substrate. It is also possible to carry out direct natural dyeing, by applying henna.

In another preferred embodiment, at least two treatment products may be applied to the nails, one of which comprises dyestuffs and the other of which comprises a film-forming compound; a nail varnish with relief effect is obtained.

In another preferred embodiment, a product comprising an enzyme, and a second product comprising a substrate, may be applied to the skin; the formation of a product in a specific place is thus obtained, with controlled kinetics.

The treatment products may also comprise additives that are common in the field under consideration, such as waxes, thickeners, surfactants, fragrances, preserving agents, pH regulating compounds, fillers and nacres.

Advantageously, each treatment product has a viscosity of between 1 and 7×10⁻³ Pa.s, at room temperature.

Each treatment product may have a surface tension of greater than 0.025 N/m and a resistivity of greater than 1 500 Ω/cm.

By means of the invention, it is thus possible to treat or make up all or a part of the human body. The treatment products may thus be in the form of, or used as, a body hygiene composition; as a hair composition, for example as a hairstyling or hair make-up composition; as a composition for making up the skin of the face, the body or the lips, for example as a lipstick, a foundation, a face powder, an eyeshadow, a fixing base to be applied over a standard lipstick, a concealer stick, a lip gloss, an eyeliner, a mascara, or temporary tattoo products; as a care composition for facial or body skin, including the scalp, the lips, the hair or the nails, for example a lipcare composition, a daily carecream or a matt-effect composition; as antisun or self-tanning composition.

By way of example, for greasy skin about 20%, and up to 25%, of ethanol by mass is included. For dry skin which allows the product to pass through more easily, a proportion of 10% ethanol by mass will be sufficient.

For a hair root treatment, the solvent may comprise water, ethanol and propylene glycol in respective proportions of from 20 to 60%, 40 to 60% and 0 to 30%, for example 22.1%, 55.1% and 22.8%. A care compound such as Minoxidil®, or nourishing components such as sugars, vitamin B6 or E, cystine, etc. may be added to the solvent.

1. A process for treating, especially for caring for, making up or coloring, at least a part of the human body, by applying at least two treatment products simultaneously to a localized area of said part of the human body as a function of characteristics of the localized area and/or of a predetermined design, in order to obtain a mixture of said treatment products directly on the localized area, at least one treatment product comprising at least one solvent in a proportion of at least 10% by weight.

2. The process as claimed in claim 1, in which the application of several treatment products is performed by jet.

3. The process as claimed in claim 2, in which at least one jet is produced by a thermal means capable of forming bubbles of treatment product.

4. The process as claimed in claim 1 or 2, in which at least one jet is produced by a piezoelectric means.

5. The process as claimed in any one of claims 2 to 4, in which at least one jet is deflected.

6. The process as claimed in any one of the preceding claims, in which a step of viewing a chosen coloration or make-up design takes place before the treatment products are applied.

7. A device for carrying out the process as claimed in any one of the preceding claims, characterized in that it comprises a means for positioning said part of the human body, and a means for applying treatment products to said part as a function of a predetermined design.

8. The device as claimed in claim 7, characterized in that the application means comprises a plurality of treatment product reservoirs and a plurality of spraying nozzles, each nozzle being fed by a reservoir.

9. The device as claimed in claim 7 or 8, characterized in that the application means comprises at least two reservoirs and/or at least two nozzles.

10. The device as claimed in any one of claims 7 to 9, characterized in that it comprises means for controlling the position of the application means relative to the part of the area to be treated.

11. The device as claimed in any one of claims 7 to 9, characterized in that it comprises means for automatic control of the total amount and of the partial amounts of each product as a function of the desired visual characteristics.
